(19) United States
(12) Patent Application Publication
(10) Pub. No.: US 2004/0054399 A1
(43) Pub. Date: Mar. 18, 2004

(54) Anti-galvanic stent coating

(76) Inventor: Noah M. Roth, Highland Park, NJ (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/245,100

(22) Filed: Sep. 17, 2002

(51) Int. Cl. A61F 2/06

(52) U.S. Cl. 623/1.16; 623/1.34

(57) ABSTRACT

A stent system is disclosed which comprises a stent made of a conventional metal alloy, such as stainless steel, coated with a nonconductive layer, in turn coated by a layer more radiodense than stainless steel, which system enhances radiopacity without permitting galvanic corrosion.

### FIELD OF THE INVENTION

The present invention generally relates to medical devices, particularly metallic and polymeric structures placed within the vasculature. More particularly, the present invention is directed to stents and/or endovascular filters that comprise a metal which enhances detectability of the device to x-rays but which does not negatively become affected with corrosion through contact of dissimilar materials (i.e., through galvanic effects).

### BACKGROUND OF THE INVENTION

Generally, stents, filters, grafts, and stent grafts are implantable medical devices (sometimes termed implantable tubular prostheses) that are placed within blood vessels and other body passageways to treat disease conditions such as stenoses, occlusions, aneurysms, and to guard against pulmonary embolism. Transluminal implantation of such devices requires that they be introduced to the site collapsed about or within an introduction device and released to self expand or, are expanded by other mechanisms to an expanded tubular state providing a lumen of approximately the same size as the patent vessel or duct lumen.

Typically, implantable devices are made from a metal alloy, such as, but not limited to, stainless steel or nitinol, and have a hollow tubular shape. To meet requirements for medical use, more fully discussed below, the devices may contain an open lattice-like structure, in which the individual metal components, such as struts, have a diameter or thickness of 0.003″ or less. This small dimension renders the strut relatively difficult to detect in techniques employing x-radiation (“x-rays”), such as fluoroscopy. Scattering of x-rays is approximately proportional to the square of atomic number, so that materials of atomic number higher than the components of the metal alloy of the stent would enhance scattering, and detectability. However, higher atomic number materials tend to be more expensive, more difficult to fabricate, and not as structurally suitable as stainless steel or nitinol.

One approach is to coat the device, comprising a typical metal alloy such as steel, with a metal of higher atomic number. However, when placing two dissimilar materials in intimate contact, there may be problems associated with corrosion through galvanic effects.

The present invention is directed to a system that has enhanced radiopacity while minimizing problems associated with galvanic effects. Before discussing this further, a review of stent use and construction is provided. Stents constructed of stainless steel will be used to describe the invention but such description is not limiting, and the invention encompasses alternate endovascular devices and/or stent materials.

When the body lumen is weakened, for example, a dissectional artery lining occurs in a body lumen such as a blood vessel, the weak part of the body lumen can inadvertently occlude a fluid passageway. To prevent such an occlusion, a stent is implanted within the blood vessel to support the blood vessel from the inside. The stent is delivered to a desired location in the blood vessel, and expanded in a circumferential direction in the blood vessel to support and maintain the patency of the blood vessel. Using the stent to support the blood vessel can avoid surgical exposing, incising, removing, replacing or bypassing a defective blood vessel required in the conventional vascular surgery.

Stents can be viewed as scaffoldings; they generally are provided with cylindrical symmetry. Stents function to physically support, and, if desired, expand the wall of the passageway. Typically, a stent consists of two or more struts or wire support members connected together into a lattice-like or open weave frame. Most stents are compressible for insertion through small cavities, and are delivered to the desired implantation site percutaneously via a catheter or similar transluminal device. Once at the treatment site, the compressed stent is expanded to fit within or expand the lumen of the passageway. Stents are typically either self-expanding or are expanded by inflating a balloon that is positioned inside the compressed stent at the end of the catheter. Intravascular stents are often deployed after coronary angioplasty procedures to reduce complications, such as the collapse of arterial lining, associated with the procedure.

There have been introduced various types of stents, and they can be typically categorized from viewpoints of methods for expanding the stent, shapes, methods for manufacturing the stent, designs and so forth. From a viewpoint of methods for expanding the stent, stents can be categorized as a self-expandable stent that can be expanded by itself, and a balloon expandable stent. In the balloon expandable stent, the stent is mounted on an expandable member, such as a balloon, provided on a distal end of an intravascular catheter, and the catheter is advanced to the desired location in the body lumen to deliver the stent. Then, the balloon on the catheter is inflated to expand the stent into a permanent expanded condition, and the balloon is deflated for removing the catheter from the stent.

Palmaz describes a variety of expandable intraluminal vascular grafts in a sequence of patents: U.S. Pat. Nos. 4,733,665; 4,739,762; 4,776,337; and 5,102,417. The Palmaz '665 patent suggests stents that are expanded using angioplasty balloons. The stents are variously a wire mesh tube or of a plurality of thin bars fixedly secured to each other. The devices are installed, e.g., using an angioplasty balloon and consequently are not self-expanding. The Palmaz '762 and '337 patents describe the use of thin-walled tubular stents with biologically compatible materials coated on stent. Finally, the Palmaz '417 patent describes the use of multiple stents or stent segments each flexibly connected to its neighbor.

In all types of stents, the stent expands from an initial diameter to a larger diameter so as to be suitable for a particular size of the targeted body cavity. Therefore, the stent must have expandability in the circumferential direction. Also, since stent is placed in the body lumen is to support a cavity wall therein to maintain the patency thereof, it is very important that the stent has radial strength as well as support capability.

At the same time, since the stent is generally delivered through tortuous path to the desired location in the body lumen, the stent must have flexibility in the axial direction. Namely, the stent must be flexible and is bent easily to thereby facilitate the delivery of the stent in the narrow and meandering body lumen.

In the aforementioned various types, since simply bending a wire creates a wire stent, generally, the wire stent is not only expanded easily, but also shrunk easily. Namely, the wire stent does not have support capability for maintaining the expanded condition in order to keep the body lumen open. On the other hand, a tubular stent generally has enough support capability to maintain its expanded condition for holding the body lumen open, and can be cut with attributes that give it the desired flexibility.

The present invention is directed to a stent system which allows the use of lower cost, more easily fabricated, stents, which are less radiodense, in conjunction with materials which are more radiodense, thereby allowing greater visualization in vivo during catheter introduction into the vessel, stent deployment, and postoperative diagnosis. Accordingly, an object of the invention is to provide a stent system which is sufficiently radiopaque, flexible, has a low profile, is substantially non-thrombogenic, and which will eliminate corrosion.

Another object of the invention is to provide an external surface in the stent system that is both biocompatible and sufficiently scattering to x-rays that the stent system is easily visualized using techniques such as fluoroscopy.

Another object of the present invention is to minimize galvanic corrosion between dissimilar metals.

### SUMMARY OF THE INVENTION

The present invention is generally directed to a stent system which comprises a material more radiopaque than the metals typically used to manufacture stents (“the radiodense material”), which radiodense material is placed within the stent system in such a way as to minimize or reduce corrosion problems associated with galvanic effects. Coating the metal of the stent with a non-electrical conducting material can minimize galvanic effects in the stent system. The radiodense material can be coated onto the non-conducting material.

One embodiment of the present invention is a stent system comprising a stent manufactured of a stainless steel coated with a non-electronically conducting layer of material, with the non-electronically conducting layer of material itself coated with a material more radiodense than stainless steel in such a way that there is no electronic contact between the stainless steel of the stent and the more radiodense material. Alternate embodiments would involve metal alloys other than stainless steel that are typically used to make stents.

A different embodiment would employ the use of an outer insulating layer than can consist of either a polymer and/or a metal.

A different embodiment would employ a plurality of non-electronically conducting and radiodense layers for purposes of providing a non-conductive layer on the outermost surface of the device. This outermost non-conductive layer can contain therapeutic agents to be delivered to the vessel intima upon implantation and expansion of the device. Alternatively the metallic interlayer can contain application of the coating that allow for the elution of therapeutic agents from the pores of an inner layer.

### BRIEF DESCRIPTION OF THE DRAWINGS

The following Detailed Description of the Invention can be better understood from the appended drawings, in which:

FIG. 1 is a depiction of the relationship between material thickness and radiodensity as it pertains to the ability to image the material under normal means.

FIG. 2 is a depiction of one embodiment of the cross-sectional arrangement of the device with its coating formulation; and

FIG. 3 is a perspective view of a device made according to the present invention.

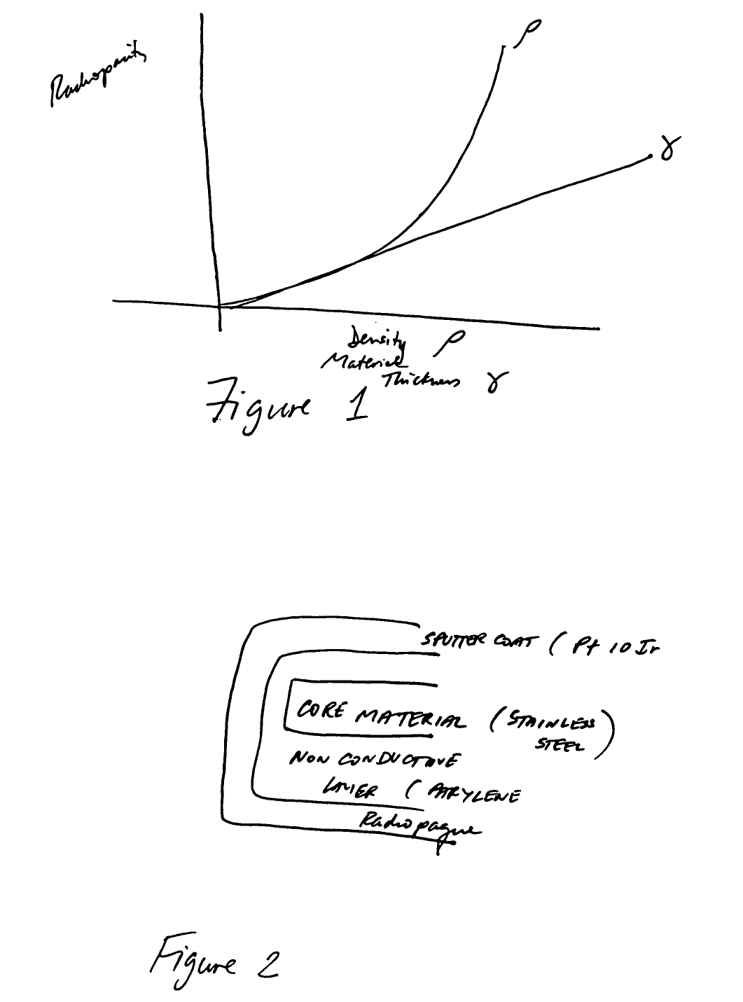

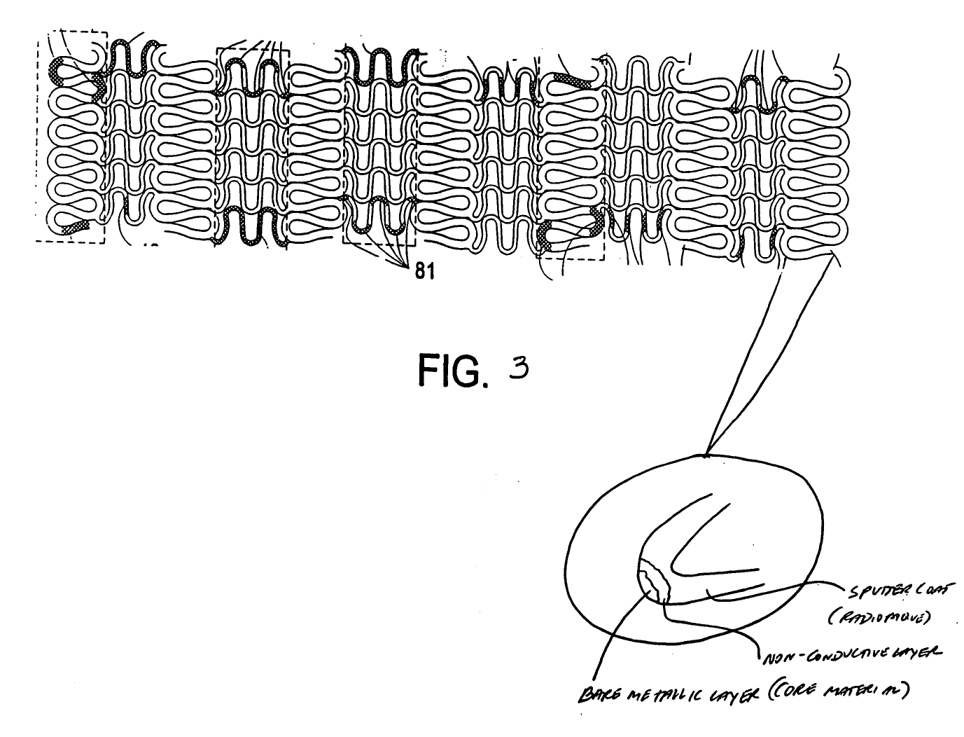

### DETAILED DESCRIPTION OF THE INVENTION

When designing endovascular medical devices (i.e., stents) there is often a need for adequate visualization (e.g., radiopacity). For example, a desired attribute of a stent is to be visualized by probes such as fluoroscopy, which employs x-rays. The ability to visualize via x-rays depends both on the scattering power of the material in the stent (scattering power going roughly as the square of the atomic number of the element; x-ray absorption also increasing with atomic number) and the amount of the material (the thicker the material, the more easily visualized).

As the density and thickness of a material increase, so does the radiopacity. However, in a move toward less invasive techniques, the thickness of a given component of a stent is limited. Further, with conventional metal alloys (e.g. stainless steel) at the dimensions used for stents (e.g., less than 0.003″), there can be difficulty in visualizing stents in fluoroscopy.

One method to resolve this is to coat (for example by sputtering) a more radiodense material (such at 90% Platinum 10% Iridium) over the stent. However, the direct contact of the metal of the stent with the metal of the radiodense material may create a galvanic effect, leading to galvanic corrosion. A method of reducing and/or removing the electrical potential across two dissimilar materials involves a precoat of the stent device with a nonconductive layer. For example, the base or core material of the stent, which might be 316L stainless steel, is coated (for example, by sputtering) with a nonconductive layer (for example, parylene), which in turn is coated (for example, by sputtering) with a more radiodense coat (for example, Pt-10 Ir).

The present invention discloses a method by which electrically conductive coatings can be applied to medical devices that are conductive in nature (e.g., metallic). The present invention overcomes problems with galvanic effects. Specifically, when two conductive materials of dissimilar electrochemical potential are in close proximity in solution (so that an electric potential is created), a galvanic effect proceeds. A galvanic effect consists of an anode (material with larger potential) and a cathode (more stable material) in which an electrical potential is created and the anode begins to degrade. In certain situations, this galvanic effect is used for electroplating of one material (cathode) with another (anode).

In the area of stents, it is often necessary to be able to visualize implantable medical devices using a fluoroscope. Visualization is a direct effect of the radiodensity of the material and of the thickness of the material being visualized. It is often desirable to use a material that has a certain characteristic (i.e., mechanical property) but may not be radiodense in the desired construction. Such devices may be coated with a secondary material that has greater radiopacity. However, as noted above, when implanted, the dissimilar charge of these materials may result in the formation of a galvanic effect that will significantly impact the corrosion resistance of the device. Alternately, the base material may be first coated with a non-conductive layer prior to being coated with the radiodense material.

A preferred embodiment of the present invention is the use of a nonconductive layer prior to coating with radiodense material. The use of this nonconductive layer makes the use of radiodense coatings both practical and safe. With the use of radiopaque coatings implantable medical devices may be manufactured smaller.

Methods of Coating

There are three main coating processes involved. First, a polymer is coated on the base metal surface. The preferred coating process is a chemical vapor deposition. This process involves the conversion of a polymer into a gaseous phase, transferred into a coating chamber, and deposition onto the base metal. Second, the radiopaque coating may be most effectively applied using a “sputter” technique with ionic assist. Sputtering is well known in the art. The ionic assist aids in providing a uniform coating more densely packs and with greater adhesion. Temperature within the sputtering process should not exceed the transition temperature of the primary polymer layer.

Alternately the polymer can be masked with a specific pattern (i.e., a dense mesh), coated with a soluble material (i.e., salt), remove the mask without damaging the primary polymer layer, and apply the radiopaque coating. After the application of the radiopaque coating is completed, the salt can be removed and a tertiary layer can be applied. The primary polymeric layer would contain therapeutic agents that can elute through the pores in the metallic coating and the last polymer layer, thus traveling into the implanted vessel.

Chemical vapor deposition is a process that transforms gaseous material into a solid in the form of thin films, typical to that used in the semiconductor industry. The process involves coupled gas-phase and gas-surface chemistry, fluid dynamics, and heat and mass transfer reactions. Ion beam technology uses the phenomenon occurring on the surface of target material, in a vacuum and under the directed flow of atomic particles. From the collision, the ions transfer their energy and momentum from the interaction ion to an atom within the target causing a cascade of energy transfer. The atoms overcome the internal forces of the target and become displaced to a new place in the structure. The use of ion beam technology in conjunction with normal sputtering techniques adds energy to the sputtered coating resulting on a more uniform coating and causes impregnation of the sputtered materials into the target resulting in a more densely packed coating layer.

A variety of embodiments have been obtained, which demonstrate the feasibility of using sputtering to coat stents.

### EXAMPLE 1

60,000 A (angstroms) gold coating with 2,000 A palladium overcoat.

### EXAMPLE 2

60,000 A gold coating with 2,000 A palladium overcoat with 2,000 A parylene overcoat.

### EXAMPLE 3

60,000 A gold coating with 2,000 A palladium undercoat.

### EXAMPLE 4

60,000 A gold coating with 2,000 A palladium undercoating with 2,000 A parylene overcoat.

### EXAMPLE 5

60,000 A gold coating.

### EXAMPLE 6

60,000 A gold coating with 2,000 A parylene overcoat.

### EXAMPLE 7

60,000 A gold plating.

In the preferred embodiments, the thickness of the radiodense material can be from 2,000 A to 80,000 A and the thickness of the nonconductive layer can be from 1,000 A to 3,000 A. The material of the nonconductive layer can be selected from the group including parylene, polyvinyl acetate, polycapralactone, urethanes, PVDF-HFP (polyvinylidenefluoride-polyhexafluoropropylene), EVA-BMA (ethylvinyl acetate-butyl methacrylate) and PHEMA-acrylic.

What is claimed is:

1. A stent comprised of stainless steel, wherein the stainless steel is coated by a non-electronically conducting material, wherein the non-electronically conducting material is coated by a material more radiodense than stainless steel.

2. The stent of claim 2 wherein the non-electronically conducting material is selected from the group consisting of parylene, polyvinylacetate, polycapralactone, urethanes, PVDF-HFP, EVA-BMA, PHEMA-acrylic, and mixtures thereof.

3. The stent of claim 1 wherein the non-electronically conducting material is parylene.

4. The stent of claim 1 wherein the radiodense material is selected from the group consisting of gold, tantalum, platinum, palladium, and iridium.

5. The stent of claim 1 wherein the coating of non-electronically conducting material is applied by a technique selected from the group consisting of dipping spraying, painting, evaporation, plasma vapor deposition, cathodic arc deposition, sputtering and ion implantation.

6. The stent of claim 1 wherein the coating of non-electronically conducting material is applied by sputtering.

7. The stent of claim 1 wherein the coating of more radiodense material is applied by a technique selected from the group consisting of dipping, spraying, painting, evaporation, plasma vapor deposition, cathodic arc deposition, sputtering and ion implantation.

8. The stent of claim 1 wherein the coating of more radiodense material is applied by sputtering.

9. The stent of claim 1 wherein the non-electronically conducting material is parylene and the more radiodense material is gold.

10. The stent of claim 1 wherein the non-electronically conducting material is parylene, which is coated to the stainless steel by sputtering and the more radiodense material is gold, which is coated to the parylene by sputtering.
